(19) United States
(12) Patent Application Publication
(10) Pub. No.: US 2015/0030571 A1
(43) Pub. Date: Jan. 29, 2015

(54) Preparation and method for producing a preparation comprising mesenchymal stem cells

(71) Applicant: Carlo Tremolada, Milano (IT)

(72) Inventor: Carlo Tremolada, Milano (IT)

(73) Assignee: LIPOGEMS INTERNATIONAL S.P.A., Milano (IT)

(21) Appl. No.: 14/382,298

(22) PCT Filed: Mar. 27, 2013
(86) PCT No.: PCT/IB2013/052462

§ 371 Date: Aug. 29, 2014

(30) Foreign Application Priority Data
Mar. 28, 2012 (IT) GE2012A000034

(51) Int. Cl. A61K 35/12; C12N 5/0775; A61K 35/28

(52) U.S. Cl. CPC A61K 35/35; A61K 35/28; C12N 5/0668
USPC 424/93.7; 435/325; 435/374; 435/283.1

(57) ABSTRACT

Preparation and method for producing a preparation or a tissue derivative that include mesenchymal stem cells, to be used in cellular therapy, for cosmetic treatments, for replacing a tissue or an organ, or inducing or accelerating tissue repair or regeneration. The method includes the steps of extracting tissue containing mesenchymal stem cells, such as adipose tissue, from a cadaveric donor by liposuction or by surgical removal of parts of adipose tissue, and mechanically treating tissue to provide a fluid component, having an oily component, a blood component and/or a sterile solution, and a solid component having cell fragments, cells and one or more cell macro-agglomerates of heterogeneous sizes, thereby separating and removing the fluid component from the solid component, which generates an emulsion of fluid components.

The present invention relates to a method for producing a preparation or a medicine comprising diploid cells, particularly mesenchymal stem cells, and to the use of said preparation in cellular therapy.

The present invention therefore is about the treatment of adult stem cells.

It is known that stem cells are found in cord blood, umbilical cord, bone marrow and in adipose tissue, particularly in the stroma-vascular fraction of the adipose tissue.

The adipose tissue is a source very rich in mesenchymal stem cells (MSCs or ASCs, adipose-derived stem cell) that is immature cells with the capability of self-renewal and differentiation into tissue-specific specialized cells.

Since they are totipotent cells, they can differentiate into cells and tissues of mesoderm origin, such as adipocytes, cartilage, musculoskeletal cells and neuronal cells.

It has also to be considered that the adipose tissue is an easily accessible tissue, a characteristic that makes it different from the bone marrow which is rich in stem cells but it is not easy to obtain.

The presence of stem cells in the adipose tissue increases the efficacy of the transplantation in cases of conservative surgery, regenerative surgery and in cases of tissue reconstruction.

The adipose tissue rich in mesenchymal stem cells therefore can be used in the field of cosmetic surgery or in regenerative surgery for restoring the anatomy and the tissue lost because of diseases, malformation or trauma.

The patent WO 2005/035742 is about a composition comprising stem cells and a method of preparing and using compositions comprising stem cells for the treatment and prevention of diseases.

The invention includes a method of preparing a purified cell population comprising stem cells to be introduced into a patient, which method provides to obtain adipose tissue from a patient, processing the adipose tissue to separate cells from other tissue components and purifying the cells separated from the other tissue components.

By purified cells it does not mean only the presence of stem cells but it means cells removed from their natural tissue environment and present at a high concentration as compared to a normal tissue environment (description at page 16).

The invention, in order to isolate cells, comprising stem cells, provides to use any tissue, not only adipose tissue.

In one embodiment the tissue is a “collagen-based tissue” such as adipose tissue or umbilical cord matrix.

The tissue can be obtained from man or animals for autologous or allogeneic use of the purified cells.

The described embodiments provide to treat the tissue with enzymes that facilitate the release of cells from other tissue components.

The method comprises one or more of the following procedures: cutting/mincing, enzymatic treatment (collagenase), ultrasonic energy treatment, perfluorocarbon treatment.

Cells can be separated from the other tissue components by known methods such as density gradient, centrifugation, filtration and a combination of said methods.

Cells prepared can be used immediately or temporarily stored prior to use (at about 4°) or they can be frozen under liquid nitrogen for long term storage.

In the description it is highlighted that purified cell populations retain viability even if stored at temperatures less than 12° and it seems that the presence of tissue components in addition to stem cells provides a therapeutic advantage, for example by promoting an appropriate differentiation (page 18).

The patent US 2010/0124776 describes a method of combining mesenchymal stem cells with a bone substrate to be used in the field of regenerative medicine. The method provides to obtain adipose tissue containing mesenchymal stem cells and other type of cells, so called unwanted cells (hematopoietic cells and stromal cells).

Stem cells obtained from the adipose tissue have been used for the induction of bone formation in tissue engineering strategies.

The adipose tissue, that can be isolated from a cadaver, is enzymatically digested such to form a cell suspension of mesenchymal stem cells and unwanted cells. The cell suspension is added to the substrate and cultured to allow the mesenchymal cells to adhere to the substrate. The substrate is then rinsed to allow unwanted cells to be removed.

The substrate can be frozen after rinsing.

The method allows an allograft to be obtained which comprises the combination of mesenchymal stem cells and a bone substrate, which allograft can be cryopreserved. The combination of the allograft with stem cells allows surgical results to be improved.

This method, that provides a chemical treatment step for the tissue extracted in vitro for obtaining the separation of stem cells from other cells and the development of mesenchymal stem cells, therefore provides to create a culture of stem cells and it is restricted to the orthopedic surgery where it is necessary to stimulate the production of new bone tissue or cartilage tissue.

The aim of the present invention is to develop a method for producing a preparation comprising mesenchymal stem cells to be used in cellular therapy, for the regenerative, therapeutic and cosmetic treatment of tissues and organs, which method allows a high amount of tissue rich in mesenchymal stem cells to be obtained in a practical, rapid and inexpensive manner, which tissue is quickly treated for obtaining a preparation, immediately usable or storable, and naturally rich in mesenchymal stem cells, which can be put in contact with the cells of the anatomical region to be treated in order to promote and accelerating the repair or regeneration of tissues/organs.

Object of the present invention is a method for preparing a preparation, a preparation comprising mesenchymal stem cells, the use of said preparation and a kit for preparing and administering said preparation.

Object of the present invention is also a device allowing high amounts of the preparation comprising mesenchymal stem cells to be produced.

A further object of the present invention is a method for therapeutic treatment providing to introduce in the body of a patient, in the anatomical region to be treated, a preparation comprising mesenchymal stem cells, which preparation can be applied alone in the site to be treated or it can be integrated on a biocompatible substrate.

Particularly the method of the present invention allows the adipose tissue, rich in mesenchymal stem cells, to be isolated by means of a closed system, that is perfectly sterile, such that it is possible to obtain, in an inexpensive and rapid way, a high quality product to be used in cellular therapy and tissue engineering.

According to the present invention the preparation method for producing a preparation or tissue derivative comprising mesenchymal stem cells, that is adult, non-embryonic stem cells, provides at least the following steps:
extraction of tissue containing mesenchymal stem cells, such as adipose tissue, from a cadaveric donor by liposuction process or by surgical removal of parts of adipose tissue,
mechanical treatment of said tissue,

said tissue, such as adipose tissue, being composed of a fluid component comprising an oily component, a blood component and/or sterile solutions and of a solid component comprising cell fragments, cells and one or more cell macro-agglomerates of heterogeneous sizes, and said mechanical treatment step being provided for separating and removing the fluid component from the solid component, which mechanical treatment step separating and removing the fluid component from the solid component provides an emulsion of fluid components to be generated, by mechanical stirring.

The emulsion is generated by using the device described in the patent application WO 2011/145075.

The device described in the international application WO2011/145075 allows acting on the tissue by mechanical forces, in a completely closed system, by providing a tissue derivative particularly a derivative of adipose tissue, highly rich in mesenchymal stem cells.

By said device the step for the mechanical treatment of the cadaveric donor-derived adipose tissue, with removal of the fluid component in the form of emulsion, can preferably provide also at least one step for reducing the sizes of the macro-agglomerates composing the tissue removed or suctioned from the cadaveric donor, into cell agglomerates with smaller sizes, such that said cell agglomerates have a size equal to or smaller than a specific value, and such that said sizes averagely are equal to each other.

Preferably the method provides the step of reducing the size of the macro-agglomerates that compose the tissue suctioned or removed from the cadaver donor followed by the step of rinsing the reduced tissue by sterile solutions and of emulsifying the fluid components of the tissue subjected to the reduction of the size of the cell macro-agglomerates.

According to the method object of the present invention in addition to the treatment of the mechanical type it is possible to provide at least one step for the chemical and/or physical treatment of the solid component obtained by reducing the size of the cell agglomerates that compose the cadaver-derived adipose tissue, by rinsing and by removing the mechanically obtained emulsion of the liquid components.

Preferably said treatment step provides to cryopreserve the solid cell component obtained by the device described in the document WO 2011/145075.

Said chemical and/or physical treatment step may allow, as an alternative or in combination one another:
selecting from said solid component only the mesenchymal stem cells to be used for transplantations in cellular therapies;
inducing the development of mesenchymal stem cells and/or differentiation of mesenchymal stem cells in a population of cells of interest depending on the anatomical region of the patient to be treated that is on the tissue or organ to be repaired, regenerated or replaced by cellular therapy treatments.

According to the present invention said solid component comprising mesenchymal stem cells or said mesenchymal stem cells selected from the adipose tissue can be integrated on at least one biocompatible graft support.

It is possible to use synthetic, natural or semi-synthetic supports (of polymer nature for the tissue reconstruction, ceramic nature for the bone reconstruction) in order to keep, replace and/or improve the function of damaged tissues or organs.

Object of the present invention is also a preparation comprising stem cells obtained by the method described above that is a preparation comprising cell fragments, cells and one or more cell agglomerates mainly composed of adipocytes and/or mesenchymal stem cells obtained by mechanically removing, in the form of emulsion, the fluid component of the cadaveric donor-derived adipose tissue.

The preparation can contain, as the cell component, exclusively mesenchymal stem cells obtained by chemically and/or physically selecting said cells from the solid component of the cadaveric donor-derived adipose tissue.

The preparation can comprise at least one biocompatible graft support upon which the cell component is integrated before or after the transplantation in the patient.

An object of the present invention is also a kit comprising cadaveric donor-derived adipose tissue and/or a preparation comprising mesenchymal stem cells obtained as described above.

The kit, besides comprising the instruments necessary for removing the adipose tissue and for applying the preparation of the present invention on the patient can also comprise at least one device as that described in patent application WO2011/145075.

For the mechanical treatment of high amounts of adipose tissue, the use of a stirrer is provided, which is disclosed with more details below, which allows several washing and separating devices to be mechanically stirred, said devices being stirred all together contemporaneously in order to obtain the emulsion of the fluid components of the cadaver-derived adipose tissue and therefore the separation, and the subsequent removal, of the fluid component from the solid component.

An object of the present invention is also the use of a preparation comprising mesenchymal stem cells obtained as described in the present application or a use of a composition comprising said preparation for first use in cellular therapy for replacing a tissue or an organ, or for inducing or accelerating tissue repair or regeneration.

An object of the present invention is also the use of a preparation comprising mesenchymal stem cells for preparing a medicine to be used in cellular therapy for replacing a tissue or an organ, or for inducing or accelerating tissue repair or regeneration.

The medicine can be used for one or more of the following treatments and/or processes:
cosmetic treatments, such as treatment of body and face volume deficiencies, improving skin trophism and/or for biological stimulation
treatment of heart diseases,
nervous system regeneration,
processes for tissue reconstruction,
processes for regenerating dental tissues comprising bone and gum,
anti-inflammatory and/or immunomodulatory processes,
revascularization/growth processes of new blood vessels,
cellular anti-apoptosis processes.

It has to be noted that the preparation of the present invention serves also for mitigating or eliminating pain.

The document WO 2005/035742, which provides only to remove the tissue from the patient and not from a cadaver, does not describe the possibility of obtaining a solid component, rich in mesenchymal stem cells, by the separation and removal of an emulsion, obtained by exclusively mechanically treating the isolated tissue.

Also the document US 2010/0124776, even if providing the removal of adipose tissue from cadaver, which tissue is enzymatically digested in order to be applied on a bone graft, it does not provide the size reduction of the adipose tissue, the washing of the reduced adipose tissue and the emulsion, obtained mechanically, of the fluid components of the adipose tissue subjected to reduction.

The document WO2011/145075 does not provide the removal from cadaveric donor and it does not provide a treatment step of the chemical and/or physical type for the solid component obtained by reducing the size of the cell agglomerates that compose the cadaver-derived adipose tissue, by washing and by removing the emulsion of fluid components.

These and other characteristics and advantages of the present invention will be more clear from the following description of some embodiments shown in the annexed drawings wherein:

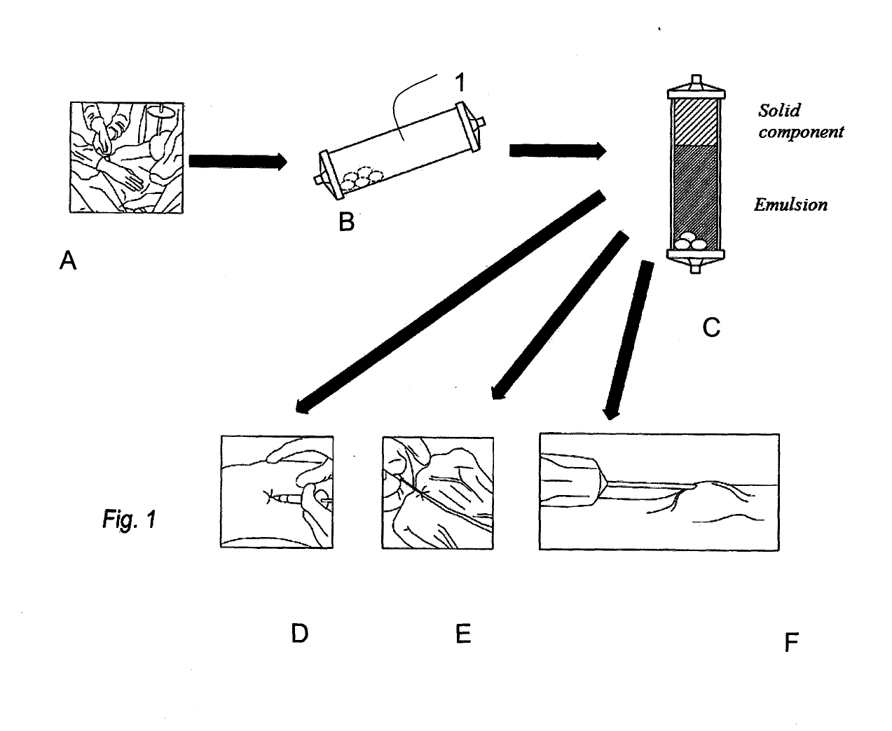

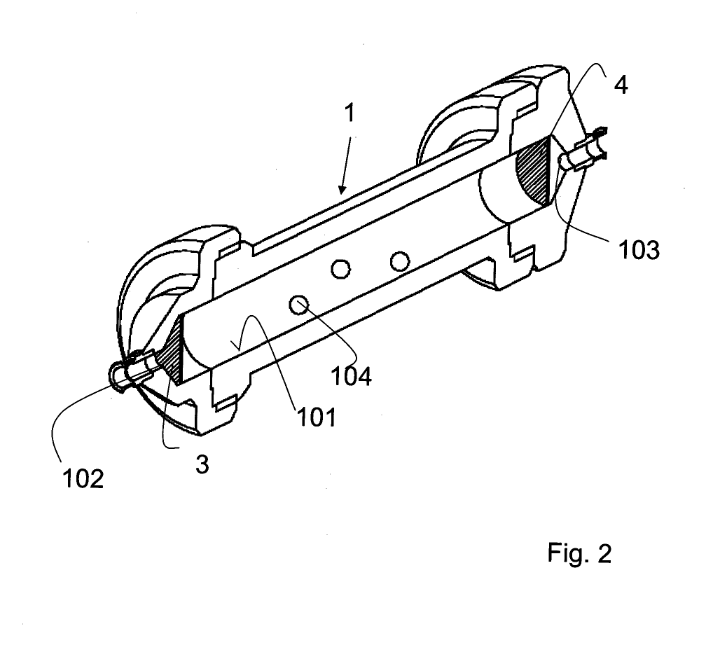

FIG. 1 schematically is the steps of extracting and using the adipose tissue from a cadaveric donor,

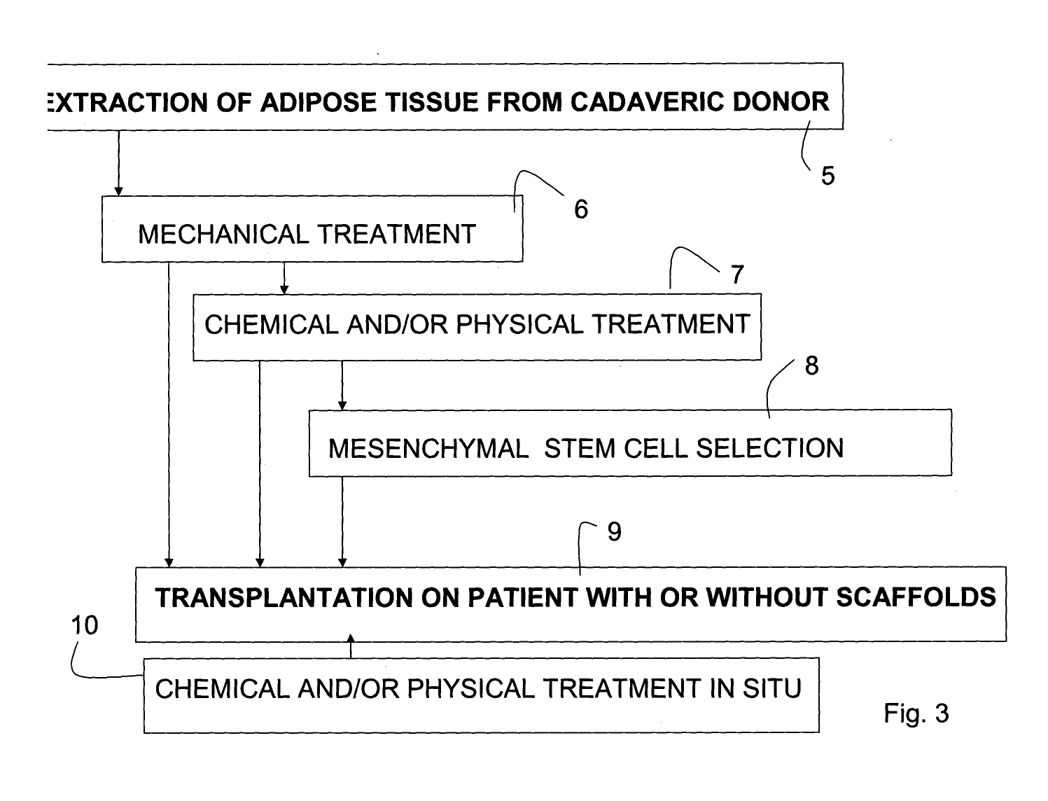

FIG. 2 is the device for the mechanical treatment of the adipose tissue,

FIG. 3 schematically is the steps of preparing and using the preparation comprising mesenchymal stem cells,

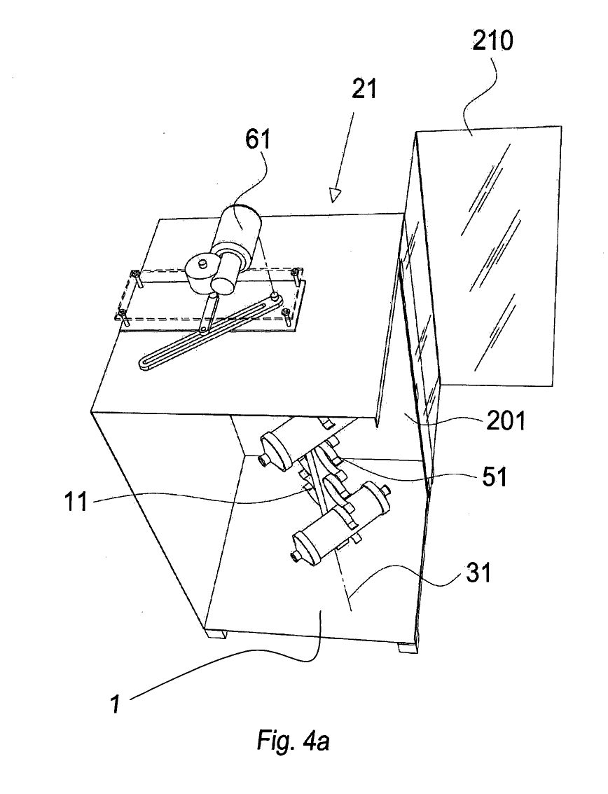

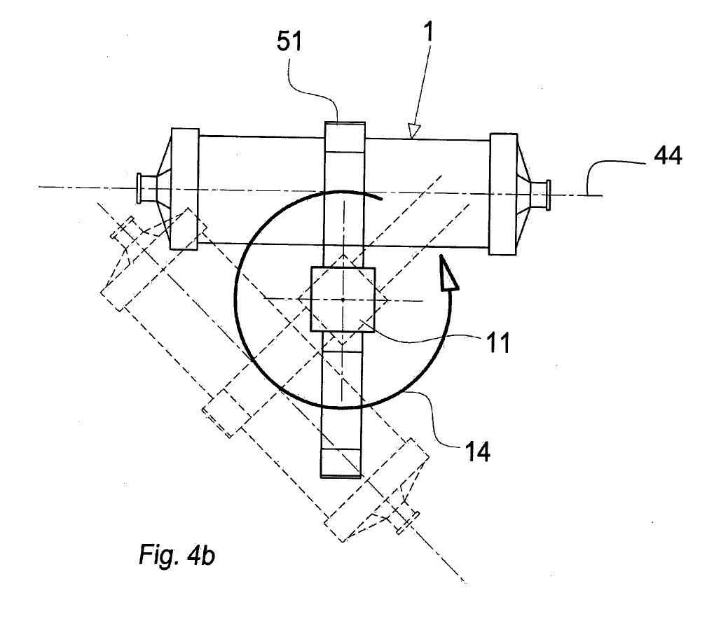

FIG. 4a is a stirrer for the mechanical treatment of high amounts of adipose tissue,

FIG. 4b is a detail of the movement of the container,

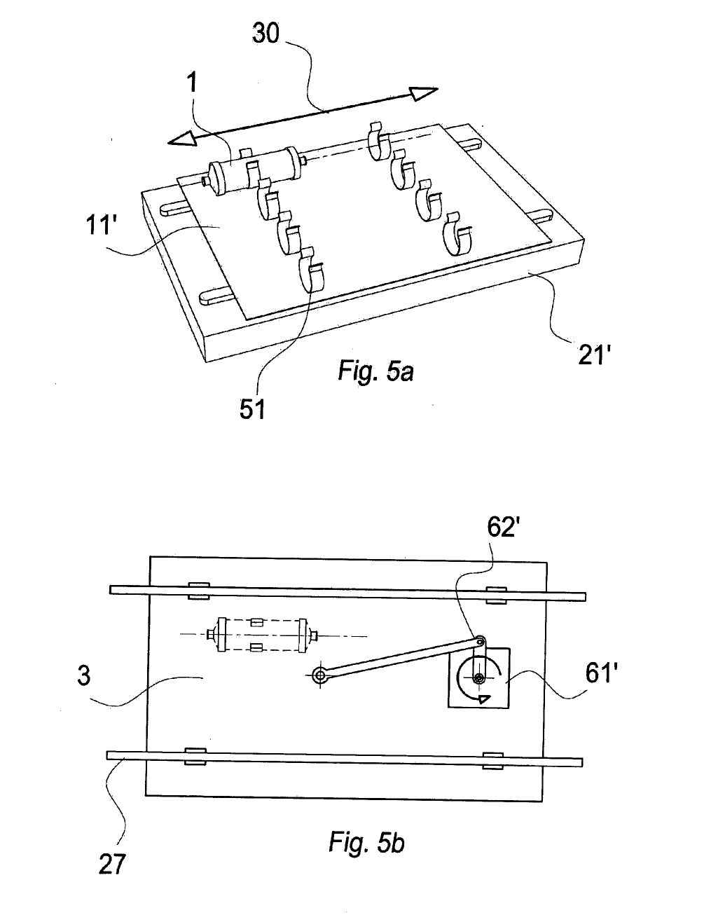

FIGS. 5a and 5b are different views of one embodiment of the stirrer,

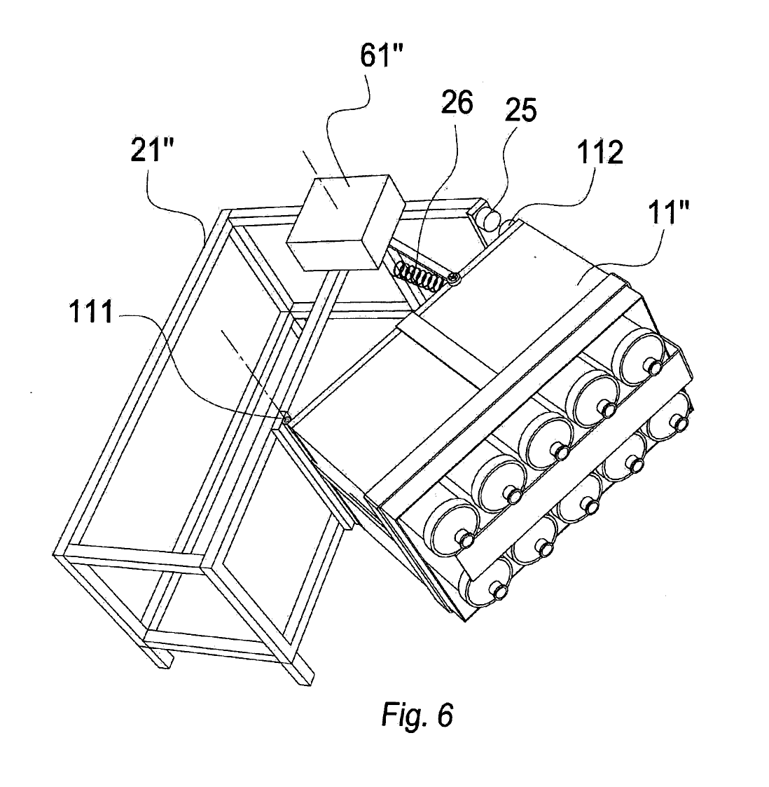

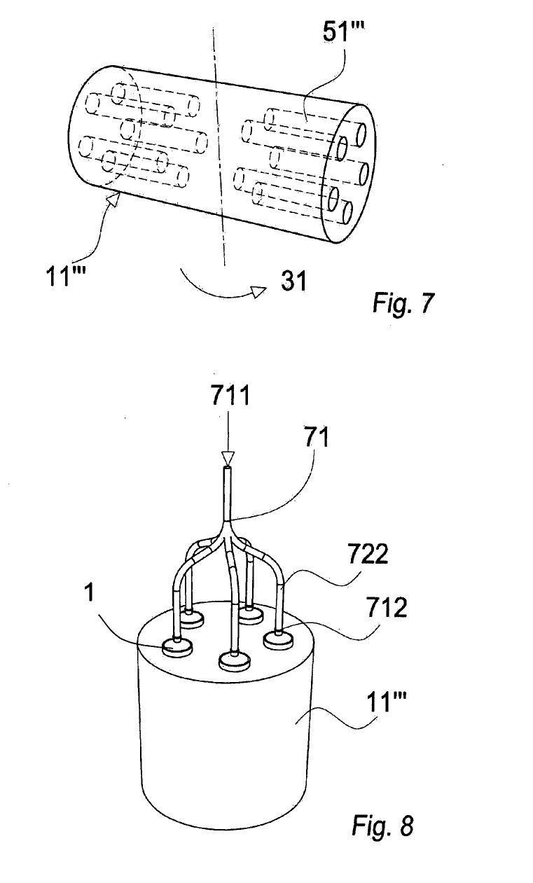

FIGS. 6 and 7 are embodiments of the stirrer,

FIG. 8 is the combination of a dispenser with a stirrer,

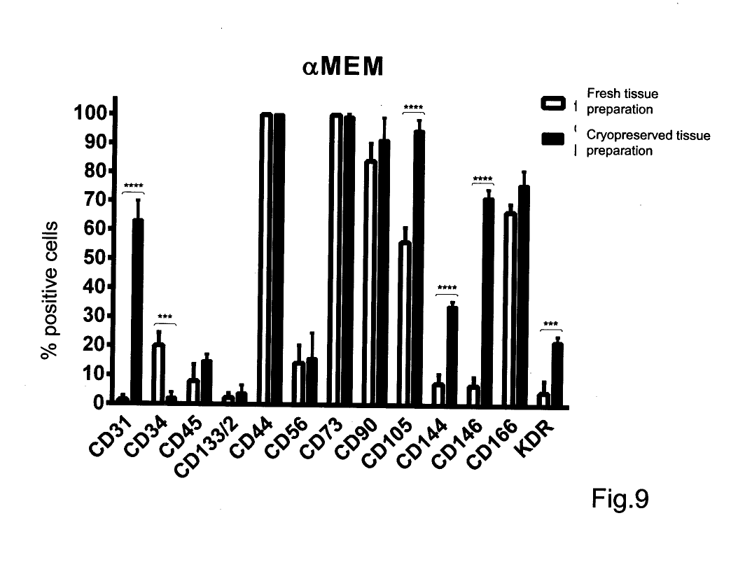

FIG. 9 is the expression of markers on αMEM medium in the tissue preparation, fresh and cryopreserved, obtained by the device shown in FIG. 2

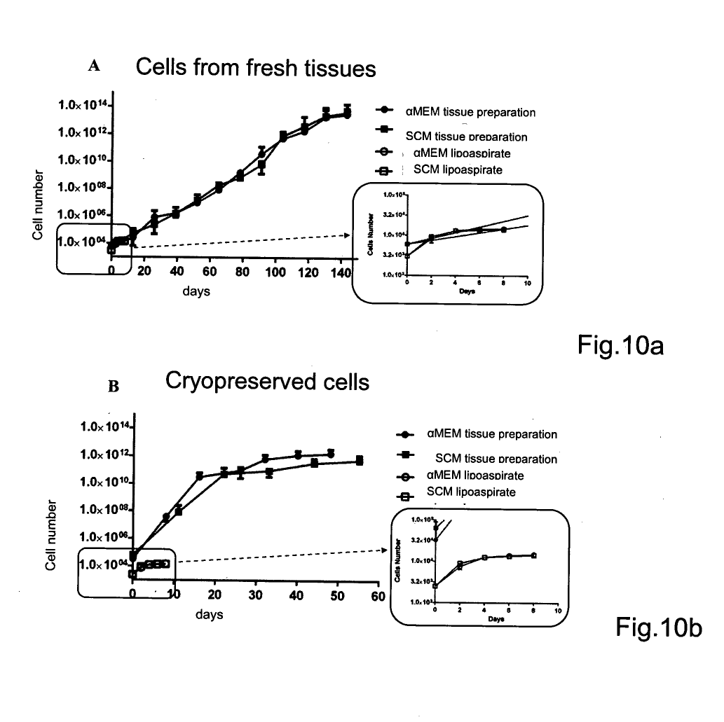

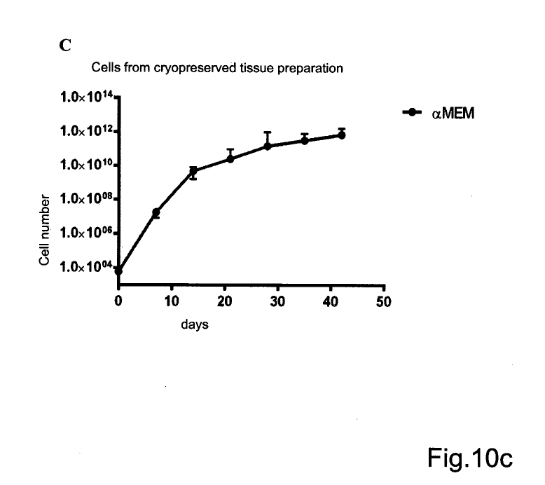

FIG. 10a is the cell growth on αMEM and SCM culture medium of non-cryopreserved lipoaspirate obtained by conventional methods and of the non-cryopreserved tissue preparation obtained by the method and device of the present invention,

FIG. 10b is the cell growth on αMEM and SCM culture medium of cryopreserved lipoaspirate obtained by conventional methods and of the cryopreserved tissue preparation obtained by the method and device of the present invention,

FIG. 10c is the cell growth on αMEM medium of mesenchymal cells from cryopreserved tissue preparation,

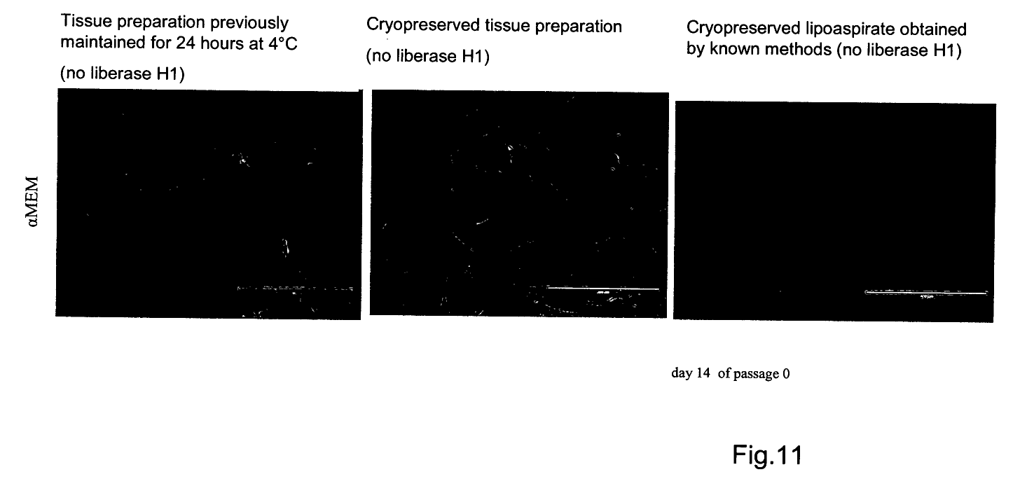

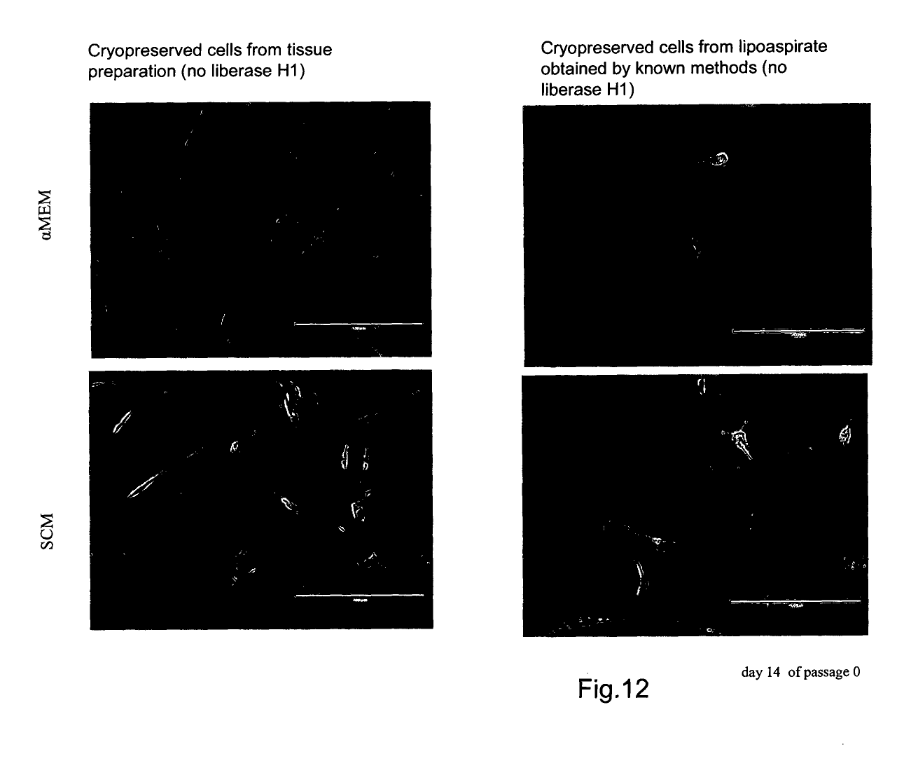

FIGS. 11 and 12 are the cell growth in the tissue preparation of the present invention with respect to a conventional lipoaspirate,

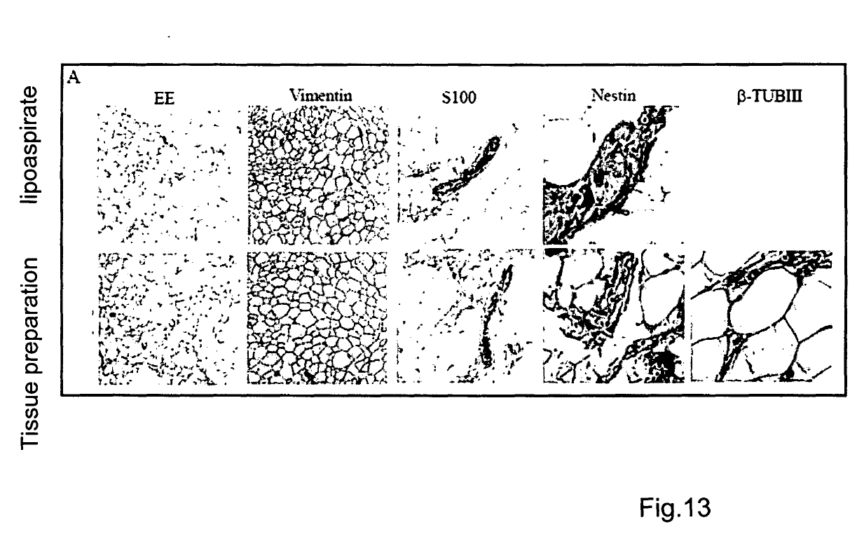

FIG. 13 is the expression of β-tubulin III in the tissue preparation of the present invention.

According to the present invention the removal of a mass or tissue containing adult stem cells that is a tissue containing mesenchymal stem cells, such as adipose tissue, is made from a cadaveric donor.

Preferably the donor is a human being but it is possible to provide the adipose tissue removal even from animal cadavers such as swine.

The removal from cadaver can be performed by a liposuction process or by the surgical removal of parts of adipose tissue.

It has been found that the adipose material removed under ischemic conditions is particularly rich in mesenchymal stem cells. Mesenchymal stem cells have shown to be more resistant to post-mortem ischemia than cells of other type. Therefore the step of the removal of the adipose tissue from a cadaver surprisingly is also a step for the selection of mesenchymal stem cells with respect to other cell types such as adipocytes or blood cells, since the step of the removal from a cadaveric donor allows a higher amount of mesenchymal stem cells by unit of volume of removed tissue to be obtained with respect to the same removal made on a living patient.

The removal from cadaveric donor allows biological material to be acquired which has an activation of the typical genes, such as HIFs, which regulate the gene transcription of a series of proteins, hormones and activities that protect against inflammatory ischemic situations, such as for example the production of erythropoietin. Therefore the stem cells collected post mortem have a higher possibility of surviving in an adverse environment such as the inflammatory ischemic one.

The procedure of liposuction from cadaver provides to clean and disinfect the area of removal and subsequently to remove the adipose tissue by means of cannulas or needles, which are provided on the surface of their shell with one or more holes for suctioning the tissue, which cannulas or needles are connected, preferably by luer lock fitting, to sterile syringes.

Obviously the volume of the suction syringe and suction cannula depends on the amount of adipose tissue available and/or on the amount of tissue to be suctioned.

According to a variant of the present invention the adipose tissue can be obtained also by surgical removal of a mass of adipose tissue.

The adipose tissue, which has been removed from a cadaveric donor according to the present invention, is rich in mesenchymal stem cells and it allows said tissue and/or a selection of mesenchymal stem cells obtained from said tissue, to be used, after mechanical treatment and preferably after chemical/physical treatment, for:
alloplastic transplantation
xenotransplantation.

FIG. 1 schematically shows what described above namely the box A shows the step of the extraction from a cadaver, while boxes D, E, F show examples of possible uses of the preparation with injection in-situ, injection in the blood flow and subcutaneous injection.

B denotes the mechanical treatment device and letter C denotes the device with the solid component therein floating on the emulsion of fluid components.

According to the present invention the adipose tissue or the mass liposuctioned and/or surgically removed from the cadaveric donor is preferably subjected to a treatment step exclusively of the mechanical type.

The mechanical treatment however can be provided in combination with a step for the chemical and/or physical treatment of said adipose tissue or mass, particularly of the cell component composed of the mesenchymal stem cells and adipocytes.

According to one embodiment of the present invention the adipose tissue liposuctioned and/or surgically removed from the cadaveric donor is treated in a device as the one described in the international patent application WO2011/145075.

The figures show the device used for treating the adipose tissue.

The device by using few and simple instruments, and in few processing steps, without using chemical substances or chemical-physical treatments, but only by mechanical stirring, allows cell agglomerates, particularly agglomerates of adipocytes and mesenchymal stem cells to be prepared, it allows contemporaneously the liquid residues mainly composed of oil and blood to be removed, avoiding also the biological material from being handled in a non perfectly sterile environment.

In one preferred embodiment of the present invention the adipose material is transferred or injected in the device, such as the one shown in FIG. 2, composed of at least one washing and separating container 1 provided with a washing chamber 101 for the adipose tissue which container 1 has an inlet 102 and an outlet 103 such that the adipose tissue removed from the cadaver can enter through the inlet 102 in the washing chamber 101 and from said chamber 101 at least a part of said tissue, particularly the fluid component, followed, after one or more washing steps, by the solid component, can exit through the outlet 103, inside said washing chamber 101 there being provided means for mechanically generating an emulsion 104 of the fluid components, on which the cell components that can be used for therapeutic purposes can float, therefore separated from the liquid component.

Said emulsion generating mechanical means are able to generate an emulsion, exclusively by a mechanical, stirring action, of blood fluids, blood residues, oils and other solutions (for example physiological washing solutions) that are comprised in the liposuctioned or removed material, allowing said fluids to remain separated from the cell solid material mainly composed of lipid cells, mesenchymal stem cells and cell fragments.

The separation of the liquid phase to be rejected from the solid phase to be transplanted occurs only by a mechanical action (not chemical). Unlike the known methods treating the adipose tissue and preparing compositions containing mesenchymal stem cells, in order to obtain a cell component highly rich in mesenchymal stem cells, the device described herein does not provide the enzymatic treatment or digestion of the tissue removed from cadaver.

Said emulsion generating means are composed of at least one stirring element 104, such as balls or the like.

The emulsion that is formed after stirring, due to the presence of mechanical emulsion means 104 inside the washing chamber 101, is discharged from the outlet 103 and preferably collected in a sealing container such not to contaminate the outer environment and at the same time such to obtain cell material (cell fragments, cells, cell agglomerates) to be transplanted stored into the washing chamber 101 under perfect sterile conditions.

Said cell material, after removing the emulsion, with or without a further chemical and/or physical treatment, is then discharged from the washing and separating chamber 101 in order to be stored, used or further chemically and/or physically treated.

The passage of the solid and fluid component that is the adipose material and/or the liquids for washing or treating said material, into the device, through the inlet and outlet, occurs by applying a pressure or suction on the contents provided in said washing and separating containers 1, that is on the material to be treated, by using compression means such as syringes connected to said containers, pistons cooperating with the openings of said containers or the like.

Inside the washing chamber 101 of the washing and separating container 1 near the outlet 103 at least one filter 4 can be provided which allows the fluid component and/or the solid component constituting the adipose material to be discharged and which allows the stirring elements 104 to be held inside the washing chamber 101.

In one embodiment said filter 4 is replaced by a sharp net of fine meshes that forms the means for reducing the size of the solid component of the removed adipose tissue, exiting from the washing chamber 101.

It has to be noted that the device object of the patent WO 2011/14507 can also allow the cell macro-agglomerates removed from cadaveric donor to be reduced in size.

According to the invention, inside the washing and separating container 1, or inside another container, defined as reducing container, fluid-tightly connectable to said washing and separating container 1, there are provided means for reducing the size of the solid component of the removed tissue, particularly cell macro-agglomerates, to averagely equal cell agglomerates, having a size reduced and equal to or smaller than a specific value, which means 3 are composed of at least one series of cutting wires or sheets arranged parallel to each other or intersecting one another such to form at least one reducing net through which the removed adipose tissue has to pass.

According to a preferred embodiment the tissue is made uniform and/or reduced in size before washing, by means of a first reducing net 3 through which the liposuctioned material has to pass which net 3 is arranged before the entrance in the washing chamber 101 of the washing and separating container 1.

Such as shown in FIG. 2 the net is arranged inside the washing chamber 101, near the inlet 102.

It is possible to provide a second size reduction/uniformity by means of a second reducing net 4, arranged before the outlet 103 in the washing chamber 101 of the washing and separating container 1, which reduction is performed after at least one cell material washing step in the washing and separating container.

The second reduction/uniformity may occur at the end of the washing, before the cell material exits from the washing and separating container 1.

The reduction serves for facilitating the washing as it breaks or stops the fibrous component of the adipose lobules and it makes the size of the cell mass uniform by reducing it into smaller agglomerates, separated from each other, with respect to the suctioned or removed adipose tissue.

Moreover the reduction allows not only to have washed cell material ready for the transplantation, of such a size that it can be injected by any type of needle, even of very small size, but above all it allows the mesenchymal stem cells to be exposed and put in higher contact both with possible substances or treatments that promote their growth, the selection or differentiation, both with the cells of the site to be treated, in case of a direct use, it being possible to apply the preparation of the present invention both alone or integrated on biocompatible supports (so-called scaffolds), after the removal from cadaver and the treatment in the device described above.

By means of the device and method object of the patent WO2011/145075 not only adipose tissue for the use as biologic filler, i.e. to be used for correcting face and body volume deficiencies is obtained but also micro-agglomerates of adipose cells naturally enriched in mesenchymal stem cells, whose arrangement, in contact with the tissue of the area wherein they are injected or applied, allows the treated tissues and/or organs to be quickly regenerated.

It has also to be noted that the preparation injected or applied on the patient performs also a function of reducing or eliminating the pain due to the release of endorphins, in the treated anatomical site, by the mesenchymal stem cells provided in said preparation.

According to the present invention it is possible to provide a stirrer that allows several washing and separating containers 1, as that described above, containing adipose tissue to be contemporaneously, manually or mechanically, stirred for the contemporaneous mechanical treatment of high amounts of tissues removed from a cadaver.

The stirrer comprises movement means for said washing and separating containers 1, means for removably coupling said washing and separating containers to said movement means wherein said movement means comprise a movement member supported by a supporting structure in a translatable manner along an axis and/or rotatable and/or pivotable about an axis of rotation parallel to the movement member or passing by the movement member, to which movement member said containers 1 are removably coupled by said coupling means, and which movement member is placed so as to rotate and/or pivot and/or translate.

The movement means can be manually driven or it is possible to provide mechanical driving means for said movement means.

Thus the tissue contained in one or more washing and separating containers 1, can be mechanically treated to obtain the generation of the emulsion and the separation of the solid component from the liquid component. Several containers 1 can be further contemporaneously moved, allowing time to be saved compared with the stirring of the individual containers, the operator being free of performing other operations and of taking the container 1 once the contents has been stirred for such a time and such a speed to form the emulsion of the fluid components.

Preferably the formation of the emulsion is performed with the container arranged horizontally with respect to the ground that is with its longitudinal axis parallel or substantially parallel to the ground, said longitudinal axis passing by the ends of the container 1, upon which the openings 102 and 103 are made.

The stirring can consist in a pivotment, or rotation of the container about an axis of rotation parallel to the movement member or passing by the movement member, a translation in two directions, alternating along a longitudinal axis or a rotation/pivotment of the container about its own longitudinal axis inside or outside the container, it being possible to provide said movements alternatively or in combination to one another.

The movement member can be a rigid rod, which rigid rod is rotated or pivoted about its own longitudinal central axis.

The movement member can be a rigid plate, which rigid plate is rotated or pivoted about an axis passing by the rigid plate itself, or an external axis and/or which rigid plate is translated along its own longitudinal axis.

As an alternative the stirring member can be composed of a cylindrical structure or a cylindrical container wherein one or more compartments housing the washing and separating containers 1 are obtained, which cylindrical structure is rotated or pivoted about an axis passing by the cylindrical structure itself, or about an external axis.

As an alternative the movement member can be composed of a basket structure comprising a plurality of housing seats for said washing and separating containers 1 which basket structure is engaged to said supporting structure by a hinge provided on a corner of said basket structure such that it is pivoted about the axis passing by said engaging corner.

Obviously it is possible to provide said cylindrical structure or basket structure to be translated alternately in both the directions along a longitudinal axis such that the container can be moved to and fro along a longitudinal axis, which longitudinal axis can be parallel to the longitudinal axis of the container 1, perpendicular to the end surfaces of the container 1.

According to one embodiment of the stirrer object of the present invention the coupling means are composed of a plurality of coupling devices fastened to said movement member and arranged on its extension and such coupling devices have such a shape and dimensions to couple to said movement member the washing and separating containers 1 having different sizes and/or they are adjustable in order to couple to said movement member the washing and separating containers 1 having different dimensions.

This allows a plurality of washing and separating containers 1 to be contemporaneously moved and it guarantees to the stirrer a great versatility and universality allowing containers 1 substantially of any shape and size to be moved.

The coupling devices can couple the washing and separating containers 1 to the movement member by elastic snap fitting or perimetral elastic tightening.

For example the coupling devices are composed of a ring element or an open ring element, with a leading and insertion opening, so called clips 51.

The driving means can advantageously be composed of an electric motor, coupable directly to the movement member or by interposing a mechanism, such as a speed variator.

Thus the movement member is moved, for example so as to rotate about an axis of rotation.

In a further embodiment the movement member is pivoted by said driving means.

In a further embodiment the movement member is translated alternately in both the directions.

In one embodiment there are provided means for switching the rotational motion to pivotal motion and/or to translation motion such that the user can select whether subjecting the movement member to a rotational or pivotal or translation motion.

It is possible to cause the movement member to vibrate by said driving means.

FIG. 4a shows an embodiment of the stirrer of the present invention, which stirrer comprises means for moving said containers 1, means for removably coupling said containers to said movement means and means for driving said movement means, wherein said movement means comprise a movement member 11, such as a rigid rod, supported by a supporting structure 21 so as to rotate or pivot about an axis of rotation 31 parallel to the movement member 11 or passing by the movement member 11, to which movement, member 11 said containers 1 are removably coupled by means of said coupling means 51.

The movement member 11 is rotated or pivoted by said driving means, which can be composed of any driving motor, particularly an electric motor 61.

It is possible to provide means for controlling the electric motor 61, for energizing and de-energizing it and for setting the rotational or pivotal speed.

The supporting structure 21 is substantially composed of a case delimiting a stirring compartment, an open side for inserting and taking the containers 1 being provided in the supporting structure.

The coupling means are composed of a plurality of coupling devices 51 fastened to the rigid rod 11 and arranged along its extension and they couple the containers 1 to the movement member 11 by elastic snap fitting or perimetral elastic tightening.

FIG. 4b shows in more detail the movement of the container 1, which container is of a substantially cylindrical shape with a longitudinal axis 44.

Each container 1 can be moved according to a rotational or pivotal path 14 such that the central longitudinal axis 44 of the container 1 is always tangent to the rotational path 14.

FIGS. 5a and 5b show different views of a further embodiment wherein the movement member 11^{I} is a plate moving alternately in opposite directions of the same axis.

Particularly the plate 11^{I} is translated alternately in one direction and in the opposite direction along an axis parallel to the longitudinal axis of at least one container coupled thereto, such as denoted by the arrow 30.

The alternate translation can be obtained by means of wheels fastened to the face of the plate 11^{I} opposite to the face for the coupling to the containers 1, which wheels engage corresponding rails provided on the supporting structure 21^{I} such to allow the plate 11^{I} to be translated.

The plate is moved by an electric motor 61^{I} by means for example of a crank-connecting rod mechanism 62^{I}, wherein the end of the connecting rod is fastened to the plate 11^{I}.

FIG. 6 shows a perspective view from the top of a stirrer wherein the movement member 11^{I} is composed of a basket structure comprising a plurality of housing seats 110 for the containers 1.

The basket structure 11^{I} is engaged to the supporting structure 21^{II} by a hinge 111 provided on a corner of the basket structure 11^{II} such that it is pivoted by an electric motor 61^{II} about the axis passing by said engaging corner.

Particularly on the supporting structure 11^{I} there are provided fixed abutments 25 placed such to define a stop position for the basket structure 11^{I} when corresponding oscillating abutments 112 fastened to the corner of the basket structure 11 opposite to the engaging corner are in contact with said fixed abutments 25.

The basket structure 11 is moved away from the stop position by the electric motor 61^{II}, that acts thereon by a crank-connecting rod mechanism fastened by one end of the connecting rod to the basket structure 11^{II}, and it is urged to return in the stop position by a spring 26, thus making an oscillating motion.

Advantageously the containers 1 composing the device for washing and separating the sizes of the cell agglomerates, are housed or retained on the movement member in a horizontal or substantially horizontal position to the ground that is with their longitudinal axis passing by the inlets and outlets 102, 103, parallel to the ground.

Obviously it is possible to provide the containers to be rotated, pivoted or translated in a vertical position with respect to the ground.

FIG. 7 shows an embodiment wherein the movement member is composed of a cylindrical structure 11^{III} in whose thickness one or more housing compartments 51^{III} for the washing and separating containers 1 are obtained, which cylindrical structure is rotated or pivoted about an axis 31 passing by the cylindrical structure, or about an external axis.

Such as shown in FIG. 8 it is possible to provide a dispenser 71 for the adipose tissue removed or suctioned from a cadaver with an inlet duct or aperture 711 for injecting the cadaver donor-derived adipose tissue which is divided into two or more ducts 722 with outlets 712 each one connectable to the inlet 102 of the washing and separating container 1 for distributing the tissue into each container 1.

The dispenser 71 can have any shape and a variable number of ducts.

The ducts can be rigid and provided in such a position that they are automatically connectable to the inlets 102 of the washing and separating container constituting the device, which containers are housed in the compartments of the movement member the arrangement of the housing compartments and therefore the position of the inlets 102 of each container being made according to a predetermined pattern coinciding with the arrangement pattern of the ducts 722 of the dispenser 71.

The combination of the dispenser 71 with the movement member provided with several compartments for housing two or more containers allows high amounts of adipose tissue to be quickly transferred and mechanically treated (with the formation of the emulsion of the liquid components).

The step of mechanical treatment of the adipose tissue removed from the cadaver therefore allows, by the generation of an emulsion, impurities to be removed, such as oil forming due to the breaking of the cell walls of the adipocytes during the mechanical steps of removing the adipose tissue from donor areas by cannulas or needles, during the passage of the adipose material, performed under pressure, through the reducing net 3.

At the end of the washing the solid component floats on the cleaned washing solution.

At the end of the washing and separating step the material contained in the chamber 101 of the container can be used or possibly further treated.

The mass of adipose tissue removed from the cadaver and prepared by the device described in the international application WO2011/145075, namely the mass subjected to a mechanical treatment reducing the sizes of the cell agglomerates and for washing it by sterile solutions, with the separation of the solid component (cells and cell fragments) from the liquid component (emulsion of oil, blood, washing liquid and/or liquids used during the removal of the tissue from cadaver) is mainly composed of adipocytes and other types of diploid cells, particularly mesenchymal stem cells.

Therefore the cell component can be used without a further treatment, for making transplantations for regenerative, cosmetic or reconstruction purposes.

The cell component obtained in this manner is a source rich in mesenchymal stem cells, immediately available for therapeutic/cosmetic purposes for the treatment and health centers.

According to the present invention, therefore the method for obtaining a preparation containing mesenchymal stem cells provides the following steps:
suctioning or removing adipose tissue from cadaveric donor,
mechanical treatment by the device and method described in patent WO2011/145075.

The preparation that is the solid component obtained from the cadaver-derived adipose tissue after at least said one mechanical treatment step in the washing and separating device described above can be:
directly used in transplantation interventions by a direct injection in the area to be treated that is it can be used for example for injections in the subcutaneous tissue, muscle tissue and in the bone, which behaves like a substrate for supporting and developing the injected cell material since it has a porous nature,
directly used for preparing grafts that is for applications on biocompatible solid or semi-solid supports (so-called scaffolds) such as ceramic porous substrates, polymer substrates, laminae, hyaluronic gel, or lyophilized human skin which substrates can be later used for transplantations for regeneration of tissues and/or organs, such as regeneration of skin, cartilage, bone or similar structures,
chemically and/or physically treated for the use in transplantation interventions with a direct injection in the area to be treated or for preparing grafts namely for applications on solid or semi-solid biocompatible supports to be grafted in the patient,
used as a starting base for isolating mesenchymal stem cells to be used alone or in combination with biocompatible supports in regenerative therapies or for starting cell cultures, particularly for cell cultures of mesenchymal stem cells, which cell cultures, one developed, according to the present invention from cadaveric donor-derived adipose tissue, can be in turn used in transplantation interventions with a direct injection in the area to be treated or for applications on solid or semi-solid supports to be used for subsequent transplantations,
subjected to a cryopreservation step.

The cryopreservation step preferably provides the solid component to be frozen at −80° C.

Particularly according to the method of the present invention in addition to the mechanical treatment it is possible to provide at least one treatment step of the chemical and/or physical type for the solid component obtained by reducing the sizes of the cell agglomerates composing the removed adipose tissue, by washing and eliminating the mechanically obtained emulsion of the liquid components.

Preferably said treatment step provides the cryopreservation of the solid component obtained by the device described in document WO 2011/145075.

Preferably the cryopreservation step is made after the mechanical treatment step.

FIG. 9 shows how the expression of stem cell markers is practically the same in the tissue preparation obtained by the method and device described above, both fresh and subjected to cryopreservation.

Surprisingly it has been found that the treatment made by freezing the solid component obtained by the method and device described above, results, after thawing it, in a more rapid cell growth, in particular of stem cells, than a simple lipoaspirate cryopreserved such as shown in FIGS. 10a, 10b and 10c.

According to the present invention the combination of the mechanical treatment of the lipoaspirate as described above, that is the reduction of the sizes of the cell macro-agglomerates with the removal of the fluid components in the form of emulsion, with the cryopreservation step allows, after thaw, the numerical expansion of stem cells.

On the contrary when a known lipoaspirate is subjected to freezing, after thawing it, the cell growth does not start again.

FIGS. 11 and 12 show how the cell growth is higher on different culture media of cells contained in conventional lipoaspirate obtained by known methods and in the preparation or tissue derivative object of the present invention.

The method object of the present invention allows a preparation rich in stem cells to be obtained avoiding the use of enzymes, such as liberase, usually used on the lipoaspirate for obtaining stem cells.

Such as shown in FIG. 13 it has been found that the cell component obtained by the method and device described above, subjected or not to the cryopreservation treatment, expresses neural antigens, (already in the tissue, without going in vitro) particularly, unlike a normal lipoaspirate obtained by known methods, the stem cells contained in the cell component express β-tubulin III i.e. they express already a neural destiny, that the lipoaspirate obtained by known methods does not have.

The expression of β-tubulin III proves that the method and device described above for the treatment of adipose tissue has an induction effect on the mesenchymal stem cells i.e. it stimulates these cells to activate and differentiate sooner and faster with respect to a known lipoaspirate in any system or site wherein they are placed.

The treatment of the adipose tissue by the method and device described above has a genic effect that is the mechanical treatment mimes what occurs in vivo with a trauma, by simulating a post-traumatic activation.

During the experimentation on the use of cadaveric donor-derived adipose tissue it has been observed that the cell component obtained from cadaver-derived adipose tissue after at least one mechanical treatment step in the washing and separating device, subjected to a cryopreservation process can be effectively used, if temperature restored, both for direct transplantations in areas to be treated and for preparing grafts and as a starting material for developing cell cultures and as a starting material for selecting mesenchymal stem cells.

Obviously it is possible to provide the cryopreservation step to be performed immediately after the step of liposuction or removal of the adipose tissue from cadaveric donor, it being possible to provide said first cryopreservation step as an alternative or in combination with a second cryopreservation step performed after the mechanical treatment step and/or a chemical/physical treatment step, if said chemical/physical treatment step is provided, for the adipose tissue.

The method object of the present invention therefore provides at least one step positioning and maintaining the cell component, alone or in combination with a graft support, in a device allowing it to be cryopreserved.

The preservation temperature for example can be of −80°.

As described above the mass or the adipose tissue mechanically treated in the device washing and separating and reducing the sizes of the cell agglomerates, particularly the solid component mainly composed of adipocytes and mesenchymal stem cells, subjected or not to cryopreservation, can be further chemically and/or physically treated such to have a preparation rich in or only composed of mesenchymal stem cells, and/or to induce the development of mesenchymal stem cells and/or differentiation of mesenchymal stem cells in a population of cells of interest depending on the region to be treated that is the tissue or organ to be regenerated or replaced.

Said at least one step of chemical and/or physical development and/or differentiation treatment can be carried out before carrying out the step of implantation on the patient, or it can be carried out in situ, that is after the implantation on the patient, by stimulating the development and/or differentiation of the mesenchymal stem cells of the complex of mesenchymal stem cells/adipocytes directly in the tissue or organ object of the treatment.

The physical treatment can be a treatment of the solid component by sound energy, light energy or the like. For example it can be placed in radiofrequency fields of Wi-Fi range.

The cell complex of adipocytes/mesenchymal stem cells treated mechanically and chemically and/or physically can be injected in situ for performing cellular therapies or it can be used for enriching the so-called scaffolds i.e. biocompatible supports, currently available on the market and for generating an integrated system of scaffolds/stem cells from preconditioned adipose tissue before the implantation or conditioned in situ, by chemical and/or physical stimuli. According to a further variant from the chemically treated cell complex of adipocytes/mesenchymal stem cells it is possible to select only the mesenchymal stem cells, without arriving to a cell expansion, to be used for transplantation, with or without integration on biocompatible supports.

The selection can be performed by the digestion of the solid cell component for forming a cell suspension having mesenchymal stem cells and cells of non interest, that can be eliminated by washing or chemical treatment.

According to a preferred embodiment of the present invention the preparation obtained after the mechanical treatment in the device described in patent WO 2011/145075 is subjected to at least one physical and/or mechanical treatment step that allows adipocytes to be eliminated and a preparation enriched with mesenchymal stem cells to be obtained.

The solid component obtained from the mechanical treatment in the washing and separating container 1, composed of adipocytes, stem cells and cell fragments can be subjected to stress such as freezing.

Other stresses that can be applied to the solid component are hypoxia and/or centrifugation, preferably pusher centrifugation.

These actions, alone or in combination one another, cause the adipocytes to break, with the formation of oil that can be easily removed for example by the device described above, but not the stem cells that therefore remain in the solid component and they allow a preparation to be obtained substantially composed only of mesenchymal stem cells.

This preparation naturally enriched with stem cells is particularly advantageous if used in cellular therapy.

Obviously it is possible to subject the selection of mesenchymal stem cells to a chemical and/or physical treatment promoting its differentiation, in vitro or in situ, into types of interest depending on the tissue or organ to be treated.

Said stem cells selected from the complex of adipocytes/mesenchymal stem cells can also be treated for obtaining a numerical expansion of the cell population.

According to the present invention therefore it is possible to treat the solid cell component, obtained by the mechanical treatment described above with substances, able to considerably increase the differentiation of the stem cells.

For example it is possible to differentiate from a cardiovascular point of view the stem cells by a treatment with esters or mixtures of hyaluronic acid with butyric acids, or other histone deacetylase inhibitors, and retinoic acids.

The treatment with such substances is also able to considerably increase the repair capacity of such cells.

According to the present invention it is also possible to treat the solid component with physical stimuli in order to obtain the differentiation of the stem cells into one or more types of cells of interest.

Said physical stimuli can be composed of radiofrequency fields of the Wi-Fi range (Radio Electric Conveyed Fields) conveyed by suitable apparatus (REAC Radio Electric Asymmetric Conveyer).

The treatment by chemical substances or physical stimuli can take place on the solid component before the transplantation on the patient or by the treatment of the area wherein the transplantation has been made.

According to the present invention therefore the complex of adipocytes/mesenchymal stem cells mechanically treated in the washing and separating device, without any further chemical/physical treatments is an optimal preparation both for interventions where it is necessary to have a cell regeneration and a filling effect, and in interventions where the filling effect is not necessary but it is sufficient for the cell component different from the mesenchymal stem cells to be a valid support for said stem cells till they are completely integrated in the treated tissue/organ.

An object of the present invention is also a kit, preferably a sterile and disposable one, to be used both in outpatient surgery and surgery field.

The kit is composed of the preparation of the present invention or by cadaveric donor-derived adipose tissue, even cryopreserved such to maintain the regenerative abilities of the mesenchymal stem cells unchanged.

Preferably the kit comprises also at least one device for washing/separating the solid component from the emulsion of the fluid components, and for the size reduction/uniformity of the cell agglomerates, said device being made as described in document WO2011/145075.

The kit object of the present invention can provide also two-, three-way fittings, with or without valves, connecting pipes, syringes, containers and needles.

The kit can provide also a biocompatible graft support upon which the cell component or only the mesenchymal stem cells can be integrated before or during the transplantation of the support in the patient.

The support is able to direct the proliferation and cell differentiation also by the controlled release of specific factors.

The kit can provide also a stirrer and/or a dispenser as described above.

The kit allows the material suctioned or removed from the cadaveric donor to be quickly treated, without risks of contaminating the cell material due to contacts with the external environment.

Therefore the kit allows biological material to be removed, the material to be treated, the biological material to be stored and injected/grafted in the patient.

An object of the present invention is also the use of the preparation described above or the use of a composition comprising said preparation for first use in cellular therapy for replacing a tissue or an organ, or for inducing or accelerating the repair or the regeneration of tissues.

From the preparation it is also possible to obtain a medicine to be used in cellular therapy for replacing a tissue or an organ, or for inducing or accelerating the tissue repair or regeneration.

Particularly the preparation can be used for:
cosmetic treatments, such as treatment of body and face volume deficiencies, improving skin trophism and/or for biological stimulation
treatment of heart diseases,
nervous system regeneration,
processes for tissue reconstruction,
processes for regenerating dental tissues comprising bone and gum,
anti-inflammatory and/or immunomodulatory processes,
revascularization/growth processes of new blood vessels,
cellular anti-apoptosis processes.

The preparation can also be used for preparing a medicine to be used in the treatments and processes described above.

The preparation of the present invention can also be used for mitigating or eliminating the pain in the patient treated with said preparation, by means of the release of endorphins by the mesenchymal stem cells.

Therefore the preparation not only is used in cellular therapy for example in cosmetic treatments, for replacing a tissue or an organ, or for inducing or accelerating the tissue repair or regeneration but it carries out also a function of mitigating or eliminating the pain at the anatomical site of the patient subjected to the treatment.

The preparation can be also used for preparing a medicine to be used for mitigating or eliminating the pain in cosmetic or therapeutic treatments that provide the use of such preparation, particularly for mitigating or reducing the pain at the anatomical site of the patient subjected to the treatment.

The therapeutic method of the present invention provides that the preparation based on mesenchymal stem cells obtained by the treatment of cadaver-derived adipose tissue can be used in cellular therapy or tissue engineering for inducing the formation and/or accelerated growth of tissues and/or organs.

In particular the preparation can be used in cosmetic and regenerative surgery.

The treatment method provides the preparation to be applied on the patient alone or in combination with biocompatible graft supports:
in cosmetic treatments, such as treatment of body and face volume deficiencies, improving skin trophism and/or for biological stimulation
in the treatment of heart diseases,
in nervous system regeneration,
in processes for tissue reconstruction,
in processes for regenerating dental tissues comprising bone and gum,
in anti-inflammatory and/or immunomodulatory processes,
in revascularization/growth processes of new blood vessels,
in cellular anti-apoptosis processes.

Obviously it is possible to provide the use of pharmaceutical substances, to be applied on the support or to be administered to the patient for promoting the integration, growth, cell regeneration and/or differentiation of the cells contained in the preparation applied on the patient.

The preparation can be used for intracutanous injections and/or intra-lesion transplantations.

According to one embodiment the method for treating pathologies provides the direct injection of the preparation in the heart.

According to a further embodiment, the preparation can be injected in the heart in a transarterial manner.

According to the present invention it is possible to provide in regenerative cardiovascular medicine the use of known scaffold devices integrated with mesenchymal stem cells of the adipose tissue, which adipose tissue is treated with synthesis molecules or with physical stimuli that increase the cardiovascular differentiation of the stem cells.

In the treatment of orthopedic pathologies or bone/cartilage tissue reconstruction the preparation of the present invention can be injected in situ in the bone of the patient or it can be used for preparing a graft to be later grafted in the patient.

The base of the graft can be composed of a support of the artificial bone type, a bone taken from the patient or a bone of a donor.

Preferably the bone constituting the base of the graft is taken from the same donor from whom the adipose tissue has been removed.

The preparation of the graft is made by treating the base directly with the preparation naturally rich in stem cells, since taken from a cadaver, without providing any step of culturing mesenchymal stem cells that can be taken from said adipose tissue.

Therefore the preparation of the present invention can:

be used alone or in combination with other substances or biocompatible supports, in cellular therapy,

be used for preparing medicines to be used in cellular therapy,

be an optimal micro-environment in vivo and in vitro for allowing the mesenchymal stem cells to act,

be used for mitigating or eliminating the pain from the areas subjected to the treatment.

The invention claimed is:

1. A method of producing a preparation or a tissue derivative comprising mesenchymal stem cells, to be used in cellular therapy, for cosmetic treatments, for replacing a tissue or an organ, or inducing or accelerating tissue repair or regeneration, the method comprising the following steps:
extracting tissue containing mesenchymal stem cells from a cadaveric donor by liposuction process or by surgical removal of parts of said tissue; and
mechanically treating said tissue,
wherein said mechanically treated tissue includes a fluid component comprising one or more of an oily component, a blood component, or a sterile solution, and a solid component comprising cell fragments, cells and one or more cell macro-agglomerates of heterogeneous sizes, and
wherein the steps of mechanically treating separates and removes the fluid component from the solid component, whereby an emulsion of fluid components is generated by mechanical stirring.

2. The method according to claim 1, the step of mechanically treating comprises using a device composed of at least one washing and separating container (1) provided with a washing chamber (101) for the tissue, the washing and separating container having an inlet (102) and an outlet (103) such that the tissue enters through the inlet (102) in the washing chamber (101) and from said chamber (101) at least a part of said tissue exits through the outlet (103), inside said washing chamber (101) there being provided means for generating, by mechanical stirring, the emulsion of the fluid components.

3. The method according to claim 2, wherein the step of mechanically treating said tissue comprises a step of reducing macro-agglomerates composing the solid component of said tissue into cell agglomerates having sizes smaller than sizes of the macro-agglomerates composing the tissue removed or suctioned from the cadaveric donor, such that said cell agglomerates have sizes equal to or smaller than a specific value, and such that said sizes on average are equal to each other, the step of reducing comprising a passage of said tissue through reducing means (3) composed of at least one series of cutting wires or sheets arranged parallel to each other or intersecting one other, such to form at least a reducing net arranged within said washing and separating container (1).

4. The method according to claim 1, further comprising a chemical or physical treatment step for the solid component, said solid component being composed mainly of the mesenchymal stem cells and adipocytes.

5. The method according to claim 4, wherein the treatment step comprises cryo-preserving the solid component.

6. The method according to claim 1, further comprising a chemical or physical treatment step for the solid component, the treatment step comprising eliminating adipocytes and maintaining the mesenchymal stem cells into the solid component, the treatment step comprising subjecting the solid component to stresses from one or more of freezing, hypoxia, or pusher centrifugation.

7. The method according to claim 1, further comprising treating the solid component chemically or physically, such to select from said solid component only the mesenchymal stem cells to be used for transplantations in cellular therapies.

8. The method according to claim 1, further comprising treating chemically or physically the solid component or the mesenchymal stem cells selected therefrom in order to induce one or more of development of the mesenchymal stem cells or differentiation of the mesenchymal stem cells in a population of cells of interest depending on an anatomical region of a patient to be treated and the tissue or organ to be repaired, regenerated or replaced by a cellular therapy treatment.

9. The method according to claim 1, further comprising the step of integrating said solid component or selected mesenchymal stem cells on at least one biocompatible graft support.

10. A preparation comprising mesenchymal stem cells, to be used in cellular therapy for cosmetic treatments, for replacing a tissue or an organ, or inducing or accelerating tissue repair or regeneration, said preparation comprising:
cell fragments, cells and one or more cell agglomerates mainly composed of one or more of adipocytes or mesenchymal stem cells obtained by mechanically removing, in form of an emulsion, a fluid component of an adipose tissue taken from a cadaveric donor,
wherein the fluid component is processed to comprise one or more of an oily component, a blood component, or a sterile solution.

11. The preparation according to claim 10, wherein said preparation contains, as a cell component, exclusively mesenchymal stem cells obtained by chemically or physically selecting said mesenchymal cells from a solid component of the adipose tissue taken from the cadaveric donor.

12. The preparation according to claim 11, wherein said preparation comprises at least one biocompatible graft support upon which the cell component is integrated before or after transplantation on a patient.

13. The preparation according to claim 10, wherein a stem cell component expresses a neural antigen.

14. A kit to be used in cellular therapy for replacing a tissue or an organ, or for inducing or accelerating tissue repair or regeneration, the kit comprising:
adipose tissue from a cadaveric donor, and
a preparation comprising mesenchymal stem cells obtained according to the method of claim 1.

15. The kit according to claim 14, wherein the kit comprises at least one device for mechanical treatment of the adipose tissue from the cadaveric donor, said adipose tissue producing a material composed of a fluid component comprising one or more of an oily component, a blood component, or sterile solutions, and a solid component comprising cell fragments, cells, and one or more cell macro-agglomerates of heterogeneous sizes,
wherein said device comprises;
a washing and separating container (1) provided with a washing chamber (101) for the adipose tissue, the container (1) having an inlet (102) and an outlet (103) such that the adipose tissue enters through the inlet (102) in the washing chamber (101) and from said chamber (101) at least a part of said material exits through the outlet (103), inside said washing chamber (101);
means for generating, by mechanical stirring, an emulsion of fluid components; and
means for reducing sizes in the solid component (3) into cell agglomerates to sizes equal to or smaller than a specific value, said means for reducing comprising a series of cutting wires or sheets arranged parallel to each other or intersecting each other to form at least one reducing net through which liposuctioned material passes at least before entering the washing chamber (101) of the washing and separating container (1).

16. The kit according to claim 15, further comprising a biocompatible graft support, upon which the solid component or only the mesenchymal stem cells are integrated before or during transplantation of the support into a patient.

17. The method according to claim 1, further comprising the step of employing the preparation or tissue derivative in a cellular therapy procedure for a cosmetic treatment, for replacing a tissue or an organ, or for inducing or accelerating tissue repair or regeneration.

18. The method according to claim 1, further comprising the step of employing the preparation or tissue derivative in a procedure for:
a treatment of body and face volume deficiencies, or for improving skin trophism and/or for biological stimulation,
treatment of heart diseases,
nervous system regeneration,
processes for tissue reconstruction,
processes for regenerating dental tissues comprising bone and gum,
anti-inflammatory or immunomodulatory processes,
revascularization/growth processes of new blood vessels, or
cellular anti-apoptosis processes.

19. The method according to claim 1, further comprising the step of employing the preparation or tissue derivative in a first medical procedure for mitigating or eliminating pain in cosmetic or therapeutic treatments.

20. The method according to claim 1, further comprising the step of employing the preparation or tissue derivative in a medication used for mitigating or eliminating pain in cosmetic or therapeutic treatments.

21. A treatment method used in cellular therapy, for cosmetic treatments, for replacing a tissue or an organ, or for inducing or accelerating tissue repair or regeneration, comprising:
injecting or grafting on a patient, alone or in combination with at least one biocompatible graft support, a preparation obtained according to claim 1.

22. The treatment method according to claim 21, further comprising the step of chemically or physically treating the preparation, along or in combination with at least one biocompatible graft support, in situ on a patient for promoting differentiation or cell expansion.

23. A stirrer configured for contemporaneously stirring a plurality of washing and separating containers (1), mechanically or manually, for mechanical treatment of adipose tissue, said stirrer comprising:
movement means for moving said washing and separating containers (1); and
coupling means for removably coupling (51, 51^{III}) said washing and separating containers (1) to said movement means,
wherein said movement means comprise a movement member (11, 11^{I}, 11^{II}, 11^{III}) supported by a supporting structure (21, 21^{II}) in a translatable way along an axis, or rotatable or pivotable about an axis of rotation parallel to the movement member or passing by the movement member, said washing and separating containers (1) being removably coupled to such movement member by said coupling means, and
wherein said movement member is configured to be rotated, pivoted, or translated.

24. The stirrer according to claim 23, further comprising a dispenser (71) for said adipose tissue into said washing and separating containers (1), said dispenser being operatively coupled to the movement member (11, 11^{I}, 11^{II}, 11^{III}) of said stirrer, said dispenser (71) being provided with an inlet duct or aperture (711), for injecting inside said dispenser, the adipose tissue extracted from a cadaveric donor, said inlet duct or aperture dividing into two or more ducts (722) with outlets (712) connectable each to an inlet (102) of one of the washing and separating containers (1) for dispensing the tissue inside each washing and separating container (1).
